(19) United States
(12) Patent Application Publication
(10) Pub. No.: US 2021/0131991 A1
(43) Pub. Date: May 6, 2021

(54) WORKPIECE AND METHOD AND SYSTEM FOR MONITORING WATER CONTENT OF LUBRICANT THEREOF, AND DETERMINATION METHOD AND APPARATUS

(71) Applicant: Schaeffler Technologies AG & Co. KG, Herzogenaurach (DE)

(72) Inventors: Jianfei Wei, Shanghai (CN); Lai Wei, Shanghai (CN); Yong Liu, Shanghai (CN); Zikui Ma, Shanghai (CN); Zhangfan Zhao, Shanghai (CN); Zhihui Pan, Shanghai (CN)

(73) Assignee: Schaeffler Technologies AG & Co. KG, Herzogenaurach (DE)

(21) Appl. No.: 16/473,818

(22) PCT Filed: Jan. 10, 2018
(86) PCT No.: PCT/CN2018/072055

§ 371 Date: Jun. 26, 2019

(30) Foreign Application Priority Data
Jan. 20, 2017 (CN) 201710046149.7

(51) Int. Cl. G01N 27/22; G01N 27/04; G01N 22/04

(52) U.S. Cl. CPC G01N 27/223; G01N 22/04; G01N 27/04

(57) ABSTRACT

Disclosed are a workpiece and a method for monitoring water content of a lubricant thereof, wherein the monitoring method comprises a water content sensing unit (10) being provided in the workpiece and exposed in a lubricant inside the workpiece, the water content sensing unit (10) and a radio frequency tag (20) being located in one and the same circuit (S1), the electrical property of the water content sensing unit (10) changing along with changes in the water content of the lubricant, and the electrical property including at least one of resistance, inductance and capacitance; determining a correlation between a frequency-related parameter of the radio frequency tag (20) and the water content in the lubricant (S2); acquiring the actual frequency of the radio frequency tag (20) (S3); and according to the correlation and the actual frequency, determining the actual water content of the lubricant (S4). The method for monitoring the water content of the lubricant of the workpiece can be realized online monitoring of the water content of a lubricant inside a bearing.

This application claims the priority of the Chinese Patent Application filed on Jan. 20, 2017, the Chinese Patent Office, Application No. 201710046149.7, entitled “Workpiece and method and system for monitoring water content of lubricant thereof, and determination method and apparatus”, the entire contents are incorporated herein by reference.

### FIELD

The invention relates to the field of bearings, in particular to a method and a system for monitoring the water content of a workpiece and a lubricant thereof, and a method and a device for determining the same.

### BACKGROUND

As a common workpiece in the mechanical field, the internal lubrication state is a key factor affecting the bearing life. Improper lubrication maintenance will lead to bearing failure, so it is necessary to monitor the lubrication state of the bearing.

Conventional monitoring methods require the bearing to be removed first, and then a portion of the lubricant in the holder and rolling element area is extracted as a sample, and the sample is tested in a chemical laboratory to obtain the water content therein. When the bearing function is good, the water content in the lubricant should be at a low level, and the water content is increased, which may result in an oil water layer in the lubricant, which may cause the lubricant to fail. Therefore, the lubrication state of the bearing can be judged based on the amount of water content.

However, the conventional monitoring method requires the bearing to be disassembled and takes a long time and has a high cost. When the bearing is installed in the service system and is in use, the lubrication state of the bearing cannot be monitored.

### SUMMARY

The problem solved by the present invention is that the conventional monitoring method cannot realize on-line monitoring, and it is necessary to disassemble the bearing, which takes a long time and a high cost.

In order to solve the above problems, the present invention provides a method for monitoring water content of a lubricant inside a workpiece, comprising: disposing a water content sensing unit in the workpiece and exposing it to a lubricant inside the workpiece, the water content sensing unit and the radio frequency label is in the same circuit, and the electrical property of the water sensing unit vary with the water content of the lubricant, the electrical property includes at least one of a resistance, and inductance, or a capacitance; determining the RF tag correspondence between the frequency-related parameter and the water content in the lubricant; acquiring an actual frequency of the RF tag; determining an actual water content of the lubricant according to the correspondence relationship and the actual frequency.

Optionally, the step of determining a correspondence between the frequency-related parameter of the RF tag and the water content of the lubricant comprises: providing a lubricant sample of the same type as the lubricant inside the workpiece; and placing the water content sensing unit In the lubricant sample, the water content sensing unit and the RF tag are disposed in the same circuit; different amounts of water are added to the lubricant sample, and one component corresponds to one of the lubricant samples. a water content; obtaining a frequency of the RF tag under each of the water content, the water content being in one-to-one correspondence with the frequency; obtaining the RF tag according to the one-to-one corresponding water content and the frequency The correspondence between the frequency and the water content of the lubricant.

Optionally, determining a correspondence between the frequency-related parameter of the RF tag and the water content in the lubricant comprises: providing a lubricant sample of the same type as the lubricant inside the workpiece; placing the water content sensing unit in the lubricant sample, the water content sensing unit is in the same circuit as the RF tag; different amounts of water are added to the lubricant sample, one component corresponding to the water content of one of the lubricant samples; obtaining a frequency of the RF tag under each of the water content, a water content corresponding to a frequency; determining a frequency difference between the obtained frequency and the set frequency, and a frequency difference corresponding to a water content, the setting of the fixed frequency is a frequency of the RF tag corresponding to the water content being a set value; and the frequency difference and the water content of the lubricant are obtained according to the one-to-one correspondence between the water content and the frequency difference.

Optionally, according to the correspondence between the frequency difference and the water content of the lubricant as well as the actual frequency, determining the water content of the lubricant comprises: comparing between the actual frequency and the set frequency Actual frequency difference; according to the correspondence between the frequency difference and the water content of the lubricant, and the actual frequency difference, the actual water content of the lubricant is obtained.

Optionally, the set frequency is: a frequency of the RF tag when the water content sensing unit is not in contact with water.

Optionally, the water content sensing unit includes at least one of a resistor, an inductor, or a capacitor.

The present invention also provides a workpiece comprising: a lubrication chamber filled with a lubricant; and a water content sensing unit that is set in the lubrication chamber and exposed to the lubricant, the water content sensing unit electrical property The electrical property includes at least one of a resistance, and inductance, or a capacitance, as the water content of the lubricant changes, and the RF tag is in the same circuit as the water content sensing unit.

Optionally, the water content sensing unit has a plurality of different positions at the lubrication chamber.

Optionally, the circuit has a plurality of, and the water content sensing units in different circuits are located at different positions of the lubrication cavity.

Optionally, the workpiece is a bearing, and the water content sensing unit is located in the lubrication cavity inside the bearing.

Optionally, the water content sensing unit has a plurality of units, each of the water content sensing units being located at different positions along a circumferential direction of the bearing.

Optionally, the circuit has a plurality of circuits, and the water content sensing units in different circuits are located at different positions along the circumferential direction of the bearing.

Optionally, the circuit is disposed on a holder of the bearing, and the water content sensing unit and the RF tag are both located on the holder.

Optionally, the retainer has a plurality of ribs arranged in a circumferential direction, and a pocket for accommodating the rolling body is formed between the circumferentially adjacent ribs; the water content sensing unit is disposed on the rib.

Optionally, the water content sensing unit is disposed on at least one of an inner circumferential surface and an outer circumferential surface of the holder.

Optionally, the circuit is formed by a coating process.

Optionally, the water content sensing unit is formed by a coating process.

Optionally, the water content sensing unit comprises a plurality of wires arranged at intervals and connected end to end.

Optionally, the water content sensing unit includes at least one of a resistor, an inductor, or a capacitor.

The present invention also provides a method for determining water content of a workpiece internal lubricant according to any one of the above, comprising: obtaining an actual frequency of the RF tag; and correspondingly according to a stored water content and a frequency related parameter of the RF tag And the actual frequency, determining the actual water content of the lubricant.

Optionally, the correspondence relationship is: a correspondence between a frequency of the RF tag and a water content of the lubricant.

Optionally, the correspondence is: a correspondence between a frequency difference between a frequency of the RF tag and a set frequency and a water content of the lubricant, where the set frequency is when the water content is a set value corresponding frequency of the RF tag.

Optionally, the step of determining the actual water content of the lubricant comprises: comparing a time frequency difference between the actual frequency and the set frequency; and according to the frequency difference and the water content of the lubricant correspondences, as well as the actual frequency difference, yield the actual water content of the lubricant.

Optionally, the set frequency is: a frequency of the RF tag when the water content sensing unit is not in contact with water.

Optionally, the method further includes: storing a correspondence between the water content and a frequency-related parameter of the RF tag.

The present invention also provides an internal lubricant water content determining apparatus according to any of the above, comprising: a frequency acquiring unit for acquiring an actual frequency of the RF tag; and a determining unit for storing the water content and frequency The correspondence, and the actual frequency acquired by the frequency acquiring unit, determines the actual water content of the lubricant.

Optionally, the correspondence relationship is: a correspondence between a frequency of the RF tag and a water content of the lubricant.

Optionally, the correspondence is: a correspondence between a frequency difference between the RF tag and the set frequency and a water content of the lubricant, where the set frequency is corresponding to the water content being a set value of the frequency of the RF tag.

Optionally, the determining unit includes: a comparing module, configured to compare an actual frequency difference between the actual frequency and the set frequency acquired by the frequency acquiring unit; and a determining module, configured to use, according to the frequency the correspondence between the difference and the water content of the lubricant, and the actual frequency difference obtained by the comparing module, results in the actual water content of the lubricant.

Optionally, the set frequency is: a frequency of the RF tag when the water content sensing unit is not in contact with water.

Optionally, the method further includes a storing unit, configured to store a correspondence between the water content and a frequency-related parameter of the RF tag.

The invention also provides a workpiece internal water content monitoring system, comprising: a water content sensing unit disposed in a workpiece and exposed to a lubricant inside the workpiece, the electrical property of the water content sensing unit changes with the water content of the lubricant, the electrical property includes at least one of a resistance, an inductance, or a capacitance; a RF tag in the same circuit as the water content sensing unit; and a RF reader for reading the radio frequency The frequency of the label; the workpiece internal lubricant water content determining device according to any one of the preceding claims, wherein the frequency acquiring unit is configured to acquire the frequency read by the RF reader.

Optionally, the water content sensing unit includes at least one of a resistor, an inductor, or a capacitor.

Compared with the prior art, the technical solution of the present invention has the following advantages:

Exposing the water sensing unit to the lubricant inside the workpiece (especially the bearing) and in the same circuit as the RF tag, when the water content of the lubricant inside the workpiece changes, the water content of sensing unit generates electricity due to contact with water, so the electrical property, such as a resistance, an inductance, or a capacitance will change, cause the frequency of the RF tag in the same circuit to change. Then, based on the correspondence between water content and frequency related parameters and the actual frequency of the RF tag the water content of the lubricant is determined. Compared with the prior art, it is not necessary to disassemble the workpiece, and even when the workpiece is installed in the service system and in use, the water content of the internal lubricant can be monitored, that is, online monitoring can be realized.

At the same time, the RF tag can adopt passive tags, and does not need to be equipped with a power supply, which can further reduce the number of electrical components, reduce wiring difficulty, and save resources.

### BRIEF DESCRIPTION OF THE DRAWINGS

FIG. 1 is a schematic diagram of a monitoring method of a first embodiment of the present invention;

FIG. 2 is a schematic structural view of a retainer of the second embodiment of the present invention when the workpiece is a bearing;

FIG. 3 is a schematic structural view of a water content sensing unit in a workpiece according to a second embodiment of the present invention; and

FIG. 4 is a schematic diagram of a monitoring system and a monitoring device thereof according to a third embodiment of the present invention.

### DETAILED DESCRIPTION

The above described objects, features, and advantages of the present invention will be more apparent from the aspects of the invention.

### First Embodiment

The embodiment provides a method for monitoring the water content of the lubricant inside the workpiece, and the workpiece can be any workpiece filled with a lubricant. For example, bearings, gear boxes, etc., are particularly suitable for monitoring the water content of the lubricant inside the bearing.

The monitoring method will be described in detail below taking the bearing as an example.

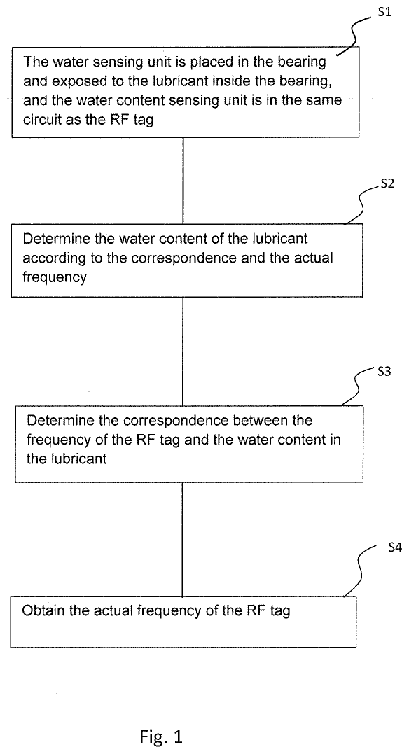

Referring to FIG. 1, the method includes the following steps S1-S4.

Step S1: The water content sensing unit is disposed in the bearing and exposed to the lubricant inside the bearing, the water content sensing unit and the RF tag are in the same circuit, and the electrical property of the water content sensing unit changes with the water content of the lubricant, the electrical property includes at least one of a resistance, an inductance, or a capacitance;

Step S2: determining a correspondence between a frequency-related parameter of the RF tag and a water content in the lubricant;

Step S3: Obtain an actual frequency of the RF tag;

Step S4: determining the water content of the lubricant according to the correspondence determined in step S2 and the actual frequency obtained in step S3.

There is no sequential relationship between step S2 and steps S1 and S3.

Those skilled in the art know that the RF tags are related to the resistor, capacitor, and inductor of the same circuit. If the electrical property of an electrical component in the circuit (including at least one of a resistor, an inductor, or a capacitor) changes, the frequency of the RF tag will also change.

In this solution, the water content sensing unit is exposed to the lubricant inside the bearing and is in the same circuit as the RF tag. When the water content of the lubricant inside the bearing changes, the electrical property, such as a resistance, an inductance, or a capacitance, of the water content sensing unit will change due to the contact with water, so the frequency of the RF tag in the same circuit will also change, and the actual frequency of the RF tag can be obtained by radio frequency communication technology. Then, the water content of the lubricant is determined based on the correspondence between the water content and the frequency and the actual frequency of the RF tag. Compared to the prior art, it is not necessary to disassemble the bearing, and even when the bearing is installed in the service system and in use, the water content of the internal lubricant can be monitored, that is, online monitoring can be realized.

Wherein, the water content sensing unit comprises at least one of a resistor, an inductor and a capacitor. When the lubricant is placed in the bearing, if the water content in the lubricant is zero or constant, the resistance value, the inductance value or the capacitance value remains stable. No change occurs. If the water content in the lubricant changes, the contact area of the water content sensing unit with water changes, resulting in a change in electrical property, such as a resistance, an inductance, or a capacitance, due to a partial short circuit. The frequency of the RF tag can be read wirelessly by means of RF communication technology. It does not need to be equipped with a power supply, which can eliminate the complicated process of wiring and does not affect the operation of the bearing.

Each step in step S1-step S4 will be described in detail below.

In step S1, the water content sensing unit is disposed in the bearing and exposed to the lubricant inside the bearing, and the water content sensing unit is in the same circuit as the RF tag.

In this step, as long as the water sensing unit is exposed to the lubricant inside the bearing, its specific position inside the bearing may not be limited, and may be, for example, a holder of a bearing, a rolling element or a raceway. However, in view of the fact that the rolling elements and the raceways are frictional contact faces, in order to prevent the water content sensing unit from being damaged by friction, a holder is preferred.

Step S2: determining the correspondence between the frequency-related parameters of the RF tag and the water content of the lubricant. The frequency-related parameter refers to a parameter related to the frequency of the RF tag, such as the frequency of the RF tag, the difference between the frequency of the RF tag and the set frequency, and the like.

In this embodiment, when determining the correspondence between the frequency-related parameters of the RF tag and the water content of the lubricant, the frequency difference of the frequency of the RF tag relative to the set frequency is used as an indication, and the water content is one-to-one correspondence. The correspondence between the two can be obtained by back-calculation. Specifically, step S2 includes the following steps S21-S24.

Step S21: Providing a lubricant sample of the same kind as the lubricant inside the bearing. Among them, theoretically, the initial water content in the lubricant sample is zero. In fact, the lubricant sample should at least meet the lubrication requirements of the bearing, and it is an unqualified product. The water content is at a low level and can be ignored.

Step S22: placing the water content sensing unit in the lubricant sample, the water content sensing unit and the RF tag are in the same circuit, and the circuit preferably includes only the closed circuit of the RF tag and the water content sensing unit. In other embodiment, some other components may be provided in the circuit, but the electrical property of the other components should be relatively stable, at least not affected by factors other than water.

Step S23: adding a different component to the lubricant sample, one component corresponding to the water content of a lubricant sample. The step 23 can be repeated in the same lubricant sample, for example, adding a predetermined dose to the lubricant in multiple times, and recording the water content after each water addition; or, step S23 can also be performed on a plurality of lubricant samples. In the process, for example, a plurality of lubricant samples are provided, and different amounts of water and the like are added to each of the lubricant samples.

Step S24: Obtain the frequency of the RF tag under each water content, and one water content corresponds to one frequency. Specifically, the relationship between the step S24 and the step S23 is repeated, and after each water filling operation is completed, before the next water adding operation, the frequency of the radio frequency label under the water content is acquired and recorded, and finally the groups are corresponding to each other. Discrete values of water content and frequency, as shown in Table 1:

TABLE 1

| Water | Corresponding water | Corresponding |
|---|---|---|
| dosage (W) | content (X) | frequency (f) |
| W1 | X1 | f1 |
| W2 | X2 | f2 |
| . . . | . . . | . . . |
| Wn | Xn | fn |

Step S25: determining a frequency difference between the acquired frequency and the set frequency, wherein a frequency difference corresponds to a water content, and the set frequency is a frequency of the corresponding RF tag when the water content is a set value.

In principle, the set value X0 of the water content is used as a reference value, and an arbitrary value can be selected. The set frequency f0 corresponding to the set value is used as the reference frequency, and then the frequency is set between the respective frequencies and the set frequency f0. So, a discrete value of the water content and frequency difference corresponding to each group is obtained. In practice, for the convenience of operation, the frequency of the corresponding RF tag when the water content sensing unit is not placed in the lubricant may be used as the set frequency f0, or the corresponding RF tag will be used when no water is added to the lubricant sample. As the set frequency f0, the corresponding water content can be regarded as 0, and the discrete values of the water content and frequency difference corresponding to each group are obtained. The list is roughly as shown in Table 2:

TABLE 2

| Water | Corresponding water | Corresponding frequency |
|---|---|---|
| dosage (W) | content (X) | difference (Δf) |
| W1 | X1 | Δf1 = (f1 − f0) |
| W2 | X2 | Δf2 = (f2 − f0) |
| . . . | . . . | . . . |
| Wn | Xn | Δfn = (fn − f0) |

Step S26: correspondence between the frequency difference and the water content of the lubricant is obtained according to the water content and the frequency difference of the one-to-one correspondence.

Specifically, after obtaining the discrete values of the frequency difference and the water content corresponding to each group, a fitting curve or a fitting function of the water content with respect to the frequency difference can be obtained as the water content and the frequency on the basis of the respective discrete values. Correspondence curve or correspondence function between the differences.

In other embodiments, step S2 may also directly use the discrete values of the frequency and water content corresponding to each group in Table 1, and obtain a fitting curve or fitting function of the water content with respect to the frequency, or frequency. A fitting curve or a fitting function of the water content is used as a correspondence curve or a correspondence function between the water content and the frequency difference, so that the above step S25 can be omitted.

Step S3: Obtain the actual frequency of the RF tag.

In step S3, the actual frequency can be obtained by wired or wireless communication.

In order not to affect the operation of the bearing and to reduce the difficulty of wiring, the embodiment adopts a wireless communication mode. For example, using radio frequency communication technology, a RF reader is used to maintain communication with the RF tag and to read the actual frequency of the RF tag. Among them, RFID (Radio Frequency Identification) and NFC (Near Field Communication) can be selected for the radio frequency communication technology. In practice, when the bearing is in the service system, the RF reader and the RF tag use the RFID chip, and the RF reader uses the RFID reader, wherein the communication distance between the RF tag and the RF reader reaches 10 meters.

Step S4: The step of determining the actual water content of the lubricant according to the correspondence relationship and the actual frequency.

In step S2, when the correspondence relationship refers to the correspondence between the frequency difference and the water content, after obtaining the actual frequency according to step S3, the difference between the actual frequency and the set frequency needs to be obtained first. The corresponding water content is obtained. Therefore, step S4 can include the following two sub-steps:

S41: Comparing the actual frequency difference between the actual frequency and the set frequency;

S42: Obtain the actual water content of the lubricant according to the correspondence between the frequency difference and the water content of the lubricant, and the actual frequency difference.

In other embodiments, when the correspondence in step S2 refers to the correspondence between the frequency and the water content, step S41 may be omitted, and the lubricant is directly obtained by the actual frequency and correspondence obtained in step S3. The actual water content.

### Second Embodiment

The embodiment provides a workpiece, which may be any workpiece having a lubrication chamber and filled with a lubricant inside the lubrication chamber, such as a bearing, a gear box, etc., and is particularly suitable for monitoring the water content of the lubricant inside the bearing.

The bearing is taken as an example for detailed introduction.

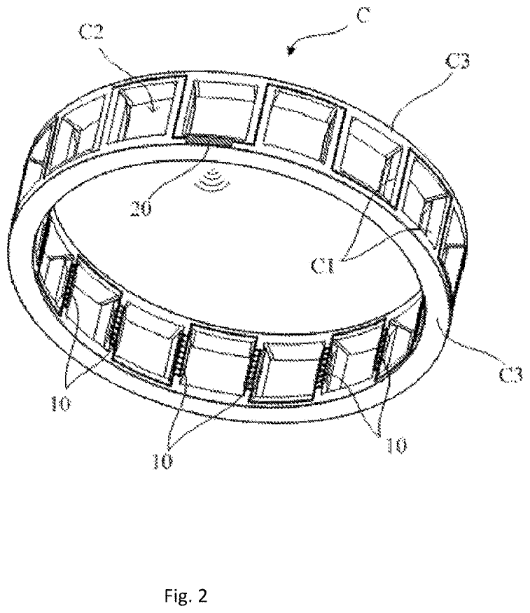

Referring to FIG. 2, the bearing is provided with a water sensing unit 10 and a RF tag 20 in the same circuit. The RF reader 30 is in wireless communication with the RF tag 20 to read the actual frequency of the RF tag 20. After reading the actual frequency, the RF reader 30 can communicate with the determining device 40 in the second embodiment to transmit the actual frequency to the determining device 40 to determine the water content of the lubricant inside the bearing by the determining device 40.

Wherein, the water content sensing unit 10 is disposed in the bearing and exposed to the lubricant inside the bearing, and the electrical property of the water content sensing unit 10 varies with the water content of the lubricant, and the electrical property includes at least one of a resistance, an inductance, or a capacitance. As an example, the water content sensing unit 10 includes at least one of a resistor, an inductor, and a capacitor, and a resistor is employed in this embodiment.

The frequency reader 30 is used to read the actual frequency of the RF tag, and the specific type of the RF reader is selected according to the communication mode and distance between the water content sensing unit 10 and the RF tag 20.

It should be understood that when the bearing is in actual operation, the water content of the lubricant will be different at different positions. During a certain period of time, the water content change at the local position may occur, while the water content in other regions is relatively unchanged. Therefore, in order to accurately monitor the water content of the lubricant in the bearing, in the present embodiment, a plurality of water content sensing units 10 are provided, and the plurality of water content sensing units 10 are circumferentially located at different positions of the bearing. Thereby, even if the water content of the lubricant at the local position changes, the frequency change of the RF tag 20 can be caused by the change in the electrical property of the water content sensing unit 10 at the corresponding position, thereby achieving accurate monitoring of the water content.

In this embodiment, the plurality of water content sensing units 10 are in the same circuit, and the RF tags 20 may change in frequency according to changes in electrical property of any one or more of the water content sensing units 10. Thereby, the apparatus can perform on-line inspection of the change in the water content in the lubricant at the first time.

In other embodiment, if it is necessary to track the specific position where the water content changes, a plurality of circuits may be provided, each of which has a water content sensing unit and a RF tag, and the water content sensing unit in the different circuits the circumferential direction is located at different positions of the bearing. The frequency of each RF tag varies with the electrical property of the water content sensing unit in the circuit, thereby monitoring the water content of the lubricant at the corresponding location.

It should be noted that, whether it is a circuit or multiple circuits, the circuit should be set as far as possible without affecting the normal operation of the bearing. Theoretically, as long as the water content sensing unit 10 is exposed to the lubricant in the bearing, the circuit can be set. There are a variety of, for example, can be placed on a rolling body, a raceway or a holder.

Referring to FIG. 2, in practice, in order to prevent the wires and their electrical components in the circuit from being damaged by friction, the circuit is disposed on the holder C of the bearing, and the water content sensing unit 10 and the RF tag 20 are used to connect the two. The wires are all located on the holder C. The water content sensing unit 10 is provided on at least one of the inner circumferential surface and the outer circumferential surface of the holder C. That is, the water content sensing unit 10 may be provided on the inner circumferential surface or the outer circumferential surface of the holder C, or the water content sensing unit 10 may be provided on both the inner circumferential surface and the outer circumferential surface. The reason for this arrangement is that the holder C is the inner circumferential surface faces the raceway of the inner ring of the bearing, and the outer circumferential surface faces the raceway of the outer ring of the bearing, and the inner ring raceway and the outer ring raceway are the main lubrication areas in the normal, which are concentrated areas of the lubricant, so providing the water content sensing unit 10 on the inner circumferential surface and the outer circumferential surface of the holder C ensures that it can be sufficiently exposed to the lubricant, thereby promptly sensing the change in the water content in the lubricant.

In order to reduce the difficulty of wiring and reduce the physical inspection of the circuit, the circuit is formed by a coating process, that is, the wires connecting the water content sensing unit 10 and the RF tag 20 in the circuit are metal films plated on the surface of the holder.

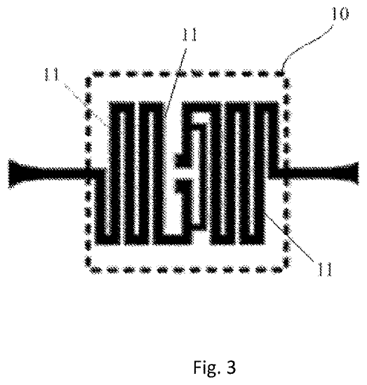

Further, the water content sensing unit 10 can also be formed by a coating process. For example, referring to FIG. 3, the water content sensing unit 10 may be a resistor formed by a plurality of spaced-apart and end-to-end wires 11 (only a part of the wires are labeled in FIG. 3), and the water content sensing unit 10 is exposed to the lubricant. In the middle, the wires 11 are all exposed to the lubricant. When the water content in the lubricant is zero, the individual wires 11 are insulated from each other; when the water content in the lubricant is greater than zero, a portion of the adjacent wires 11 will be short-circuited due to contact with water, resulting in a resistance value. When the water content in the lubricant increases, the area where the short circuit occurs in the water content sensing unit 10 increases, and the resistance value changes accordingly. In other embodiment, the water content sensing unit may also be an inductor and capacitor.

As shown in FIG. 2, the holder C has a plurality of ribs C1 arranged in the circumferential direction, and a pocket C2 for accommodating the rolling elements is formed between the circumferentially adjacent ribs C1, and only some of the ribs and pockets are formed in FIG. 3 are labeled. The water content sensing unit 10 is disposed on the rib C1. In the axial direction of the holder C, the position of the rib C1 is in the middle of the holder C with respect to the ring member C3 on both sides of the rib C1, and the lubricating oil is more concentrated. It is further ensured that the water content sensing unit 10 is sufficiently exposed to the lubricant.

In other embodiment, when the workpiece is a component other than the bearing, the circuit can also be placed in a non-friction area inside the lubrication chamber of the workpiece by a coating process to ensure the performance and service life of the monitoring device.

### Third Embodiment

The embodiment is based on the workpiece according to the second embodiment, and provides a method for determining the water content of the lubricant inside the workpiece, the method comprising the following steps T1-T2;

Step T1: Obtain an actual frequency of the RF tag;

Step T2: determining the actual water content of the lubricant according to the correspondence between the stored water content and the frequency-related parameters of the RF tag, and the actual frequency.

In step T2, the correspondence relationship may be: a correspondence between the frequency of the RF tag and the water content of the lubricant. The correspondence relationship may be: a correspondence between a frequency difference between the frequency of the RF tag and the set frequency and a water content of the lubricant, and the set frequency is a frequency of the corresponding RF tag when the water content is a set value. The determination method of the two correspondences and the manner of selecting the set frequency can be referred to the first embodiment.

### Third Embodiment

The embodiment is based on the workpiece according to the second embodiment, and provides a method for determining the water content of the lubricant inside the workpiece, the method comprising the following steps T1-T2;

Step T1: Obtain an actual frequency of the RF tag;

When the correspondence relationship is the correspondence between the frequency difference and the water content of the lubricant, step T2 may further include the following steps T21 to T22:

Step T21: Comparing the actual frequency difference between the actual frequency and the set frequency;

Step T22: correspondence between the frequency difference and the water content of the lubricant, and the actual frequency difference. The actual water content of the lubricant is obtained.

In other embodiments, when the correspondence in step T2 refers to the correspondence between the water contents, steps T21 to T22 may be omitted, and the lubricant is directly obtained according to the actual frequency and correspondence obtained in step T1 for actual water content.

It should be understood that, in order to complete step T2, the method of this embodiment may further include the following step T3: storing the correspondence between the water content and the frequency-related parameters of the RF tag. There is no relationship between T3 and T1, but it should be completed before step T2.

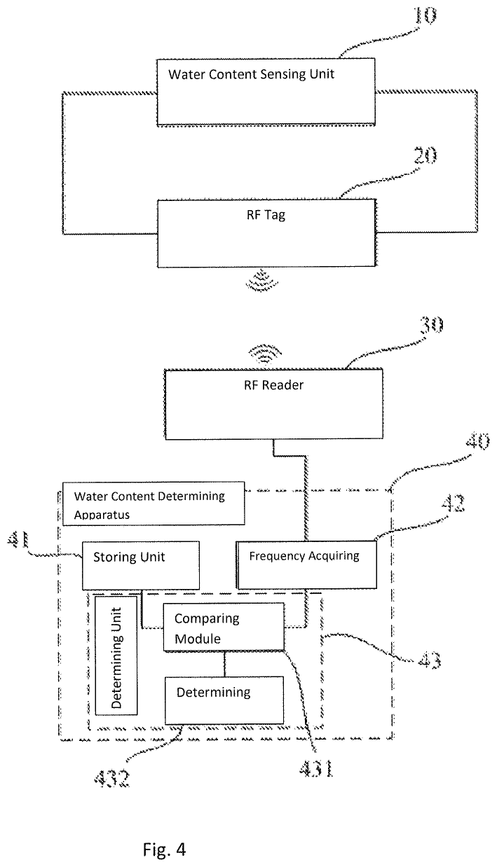

Referring to FIG. 4, the embodiment further provides a water content monitoring system for the internal lubricant of the workpiece and a water content determining device 40. In the monitoring system, when monitoring the water content of the lubricant inside the workpiece, it is required that the water content sensing unit 10 is disposed inside the workpiece, and the RF tag 20 is in the same circuit as the water content sensing unit 10. The RF tag 20 is in wireless communication with the RF reader 30, and the frequency of the RF tag 20 is read by the RF reader 30. Among them, the characteristics and types of the water content sensing unit 10 are the same as those of the first embodiment.

The RF reader 30 transmits the read frequency signal to the water content determining device 40, and the water content determining device 40 determines the water content in the lubricant based on the frequency signal acquired from the RF reader 30.

Specifically, the water content determining device 40 includes a storing unit 41, a frequency acquiring unit 42, and a determining unit 43.

The storing unit 41 is configured to store the correspondence between the water content of the lubricant and the frequency-related parameters of the RF tag. The correspondence relationship may be: the correspondence between the frequency of the RF tag and the water content of the lubricant. Alternatively, the correspondence relationship may be: a correspondence between a frequency difference between the frequency of the RF tag and the set frequency and a water content of the lubricant, and the set frequency is a frequency of the corresponding RF tag when the water content is a set value, wherein the two corresponding The manner of determining the relationship and the method of selecting the set frequency can be referred to the first embodiment.

The frequency acquiring unit 42 is configured to acquire the actual frequency of the RF tag. The determining unit 43 determines the actual water content of the lubricant based on the correspondence between the water content of the storing unit 41 and the frequency-dependent parameter of the RF tag, and the actual frequency acquired by the frequency acquiring unit 42.

When the correspondence relationship is the correspondence between the frequency difference value and the water content of the lubricant, then correspondingly, the storing unit 41 stores the correspondence relationship between the frequency difference value and the water content. Wherein the set frequency is the frequency of the corresponding RF tag when the water content is the set value, preferably the frequency of the RF tag when the water content of the lubricant is 0, for example, the RF tag 20 when the water content sensing unit 10 is not placed in the lubricant.

When the correspondence relationship is the correspondence between the frequency difference and the water content of the lubricant, the determining unit 43 may further include a comparing module 431 and a determination module 432.

The comparing module 431 is configured to compare the actual frequency difference between the actual frequency and the set frequency obtained by the frequency acquiring unit, and the determining module 432 is configured to use the correspondence between the frequency difference and the water content of the lubricant, and the comparing module. Actual frequency difference shows the actual water content of the lubricant.

When the correspondence relationship is the correspondence between the frequency difference value and the water content of the lubricant, then correspondingly, the storing unit 41 stores the correspondence relationship between the frequency difference value and the water content. Wherein the set frequency is the frequency of the corresponding RF tag when the water content is the set value, preferably the frequency of the RF tag when the water content of the lubricant is 0, for example, the RF tag 20 when the water content sensing unit 10 is not placed in the lubricant.

In other embodiments, when the correspondence relationship refers to the correspondence between the frequency of the RF tag and the water content, the correspondence relationship between these in the storing unit 41 is the relationship between the frequency of the RF tag and the water content. Then, the comparing module 431 can be omitted, and the actual water content of the lubricant can be obtained directly according to the actual frequency obtained by the frequency acquiring unit 42 and the storing unit 41 storing the correspondence.

Although the present invention has been disclosed above, the present invention is not limited thereto. The scope of the present invention should be determined by the scope of the claims, and the scope of the invention is defined by the scope of the appended claims.

1. A method for monitoring water content of a lubricant inside a workpiece, comprising:
disposing a water sensing unit in the workpiece and exposing the water sensing unit to a lubricant inside the workpiece, the water sensing unit being in a same circuit as an RF tag, an electrical property of the water sensing unit changing with the water content of the lubricant, the electrical property includes at least one of a resistance, an inductance, or a capacitance;
determining a correspondence between a frequency-related parameter of the RF tag and a water content in the lubricant; and
obtaining an actual frequency of the RF tag.

2. The method of claim 1, wherein the step of determining a correspondence between a frequency dependent parameter of the RF tag and a water content of the lubricant comprises:
providing a lubricant sample of a same type as the lubricant inside the workpiece;
placing a water content sensing unit in the lubricant sample, and setting the water content sensing unit and the RF tag in the same circuit;
adding different amounts of water to the lubricant sample, one component corresponding to a water content of the lubricant sample;
obtaining a frequency of the RF tag for each of the different amounts of the water content, such that the water content and the frequency are in one-to-one correspondence; and
obtaining the correspondence between the frequency of the RF tag and the water content of the lubricant according to the water content and the frequency based on the one-to-one correspondence.

3. The method of claim 1, wherein determining a correspondence between a frequency dependent parameter of the RF tag and a water content in the lubricant comprises:
providing a lubricant sample of the same type as the lubricant inside the workpiece;
placing a water content sensing unit in the lubricant sample, wherein the water content sensing unit is in a same circuit as the RF tag;
obtaining a frequency of the RF tag for each of the different amounts of the water content, determining a frequency difference between the obtained frequency and a set frequency, wherein the frequency difference corresponds to a water content; and the set frequency is the frequency of the RF tag corresponding to the value;
obtaining the correspondence between the frequency difference and the water content of the lubricant according to the water content and the frequency difference of the one-to-one correspondence.

4. The method according to claim 3, wherein the step of determining the water content of the lubricant based on the correspondence between the frequency difference and the water content of the lubricant and the actual frequency comprises:
comparing an actual frequency difference between the actual frequency and the set frequency;
obtaining the actual water content of the lubricant based on the correspondence between the frequency difference and the water content of the lubricant, and the actual frequency difference.

5. The method according to claim 4, wherein the set frequency is a frequency of the RF tag when the water content sensing unit is not in contact with water.

6. The method of claim 1, wherein said water sensing unit comprises at least one of a resistor, an inductor, or a capacitor.

7. A workpiece comprising:
a lubrication chamber filled with a lubricant;
a water content sensing unit disposed in the lubrication chamber and exposed to the lubricant,
wherein an electrical property of the water sensing unit changes with a water content of the lubricant, the electrical property includes at least one of a resistance, an inductance, or a capacitance, and
an RF tag in a same circuit as the sensing unit.

8. The workpiece of claim 7, wherein said water sensing unit has a plurality of sensor locations located at different locations of said lubrication chamber.

9. The workpiece of claim 7, wherein said workpiece has a plurality of said water content sensing units in different circuits located at different locations of said lubrication chamber.

10. A workpiece according to claim 7, wherein said workpiece is a bearing, and said water content sensing unit is located in said lubrication chamber inside said bearing.

11. The workpiece according to claim 10, wherein said water content sensing unit has a plurality of said water content sensing units located at different positions along a circumferential direction of said bearing.

12. A workpiece according to claim 10, wherein said circuit has a plurality of said water content sensing units in different circuits located at different positions along a circumference of said bearing.

13. A workpiece according to claim 10, wherein said circuit is provided in a holder of said bearing, said water sensing unit and said RF tag being located on said holder.

14. The workpiece according to claim 13, wherein said holder has a plurality of ribs arranged in a circumferential direction, and pockets for accommodating rolling elements are formed between circumferentially adjacent ribs; and the water content sensing unit is set on the ribs.

15. The workpiece according to claim 13, wherein said water content sensing unit is provided on at least one of an inner circumferential surface and an outer circumferential surface of said holder.

16. The workpiece of claim 7, wherein said circuit is formed by a coating process.

17. The workpiece according to claim 7, wherein said water content sensing unit is formed by a coating process.

18. The workpiece according to claim 17, wherein said water content sensing unit comprises a plurality of spaced-apart and end-to-end wires.

19. The workpiece according to claim 7, wherein said water content sensing unit comprises at least one of a resistor, an inductor, and a capacitor.

20. A method for determining a water content of an internal lubricant of a workpiece, comprising:
obtaining an actual frequency of an RF tag that is in communication with a water content sensing unit in a lubrication chamber of the workpiece in real time;
determining the actual water content of the lubricant based on a correspondence between stored water content and frequency dependent parameters of the RF tag, and the actual frequency.

21. The method of claim 2Q wherein said correspondence is a correspondence between a frequency of said RF tag and a water content of said lubricant.

22. The method according to claim 20, wherein the correspondence relationship is: a correspondence between a frequency difference between a frequency of the RF tag and a set frequency and a water content of the lubricant, the set frequency is the frequency of the RF tag corresponding to a set value.

23. The method of claim 22, wherein the step of determining the actual water content of said lubricant comprises:
comparing the actual frequency difference between the actual frequency and the set frequency; and
obtaining the actual water content of the lubricant based on the correspondence between the frequency difference and the water content of the lubricant, and the actual frequency difference.

24. The method according to claim 22, wherein said set frequency is a frequency of said RF tag when said water content sensing unit is not in contact with water.

25. The method of claim 22, further comprising:
storing a correspondence between said water content and a frequency dependent parameter of said RF tag.

26. An apparatus for determining an internal lubricant water content of a workpiece, comprising:
a frequency acquiring unit, configured to acquire an actual frequency of an RF tag;
a determining unit configured to correspond to a frequency-related parameter of the RF tag according to a stored water content, and an actual frequency obtained by the frequency acquiring unit to determine an actual water content of the lubricant.

27. The apparatus according to claim 26, wherein said correspondence relationship is a correspondence between a frequency of said RF tag and a water content of said lubricant.

28. The device according to claim 26, wherein said correspondence relationship is: a correspondence between a frequency difference between said RF tag and a set frequency and said water content of said lubricant, said set frequency being the frequency of the corresponding RF tag when the set value is set.

29. The apparatus according to claim 28, wherein said determining unit comprises:
a comparing module, configured to compare an actual frequency difference between the actual frequency acquired by the frequency acquiring unit and the set frequency;
and a determining module, configured to obtain an actual water content of the lubricant according to a correspondence between the frequency difference and the water content of the lubricant, and the actual frequency difference obtained by the comparing module.

30. The apparatus according to claim 28, wherein said set frequency is a frequency of said RF tag when said water content sensing unit is not in contact with water.

31. The apparatus according to claim 26, further comprising a storing unit for storing a correspondence relationship between said water content and a frequency of said RF tag.

32. A workpiece internal lubricant water content monitoring system, comprising:
a water content sensing unit provided in a lubricant in the workpiece and exposed inside the workpiece, an electrical property of the water content sensing unit changing with a water content of the lubricant, the electrical property includes at least one of a resistance, an inductance, or a capacitance;
a RF tag, in a same circuit as the water content sensing unit; and
a RF reader for reading a frequency of the RF tag.

33. The workpiece internal lubricant water content determining apparatus according to claim 26, further comprising a frequency acquiring unit configured to acquire a frequency read by the RF reader.

34. The system of claim 32, wherein said water content sensing unit comprises at least one of a resistor, an inductor, and a capacitor.
